(19) United States
(12) Patent Application Publication
(10) Pub. No.: US 2010/0056983 A1
(43) Pub. Date: Mar. 4, 2010

(54) Treatment of cancer using photodynamic therapy

(75) Inventors: Thomas J. Dougherty, Grand Island, NY (US); Ravindra K. Pandey, Williamsville, NY (US); William R. Potter, Amherst, NY (US)

Correspondence Address: MICHAEL L. DUNN, SIMPSON & SIMPSON, PLLC, 5555 MAIN STREET, WILLIAMSVILLE, NY, 14221, US

(73) Assignee: Health Research, Inc., Buffalo, NY (US)

(21) Appl. No.: 12/462,606

(22) Filed: Aug. 6, 2009

(30) Foreign Application Priority Data
Sep. 27, 2007 (US) PCT/US07/20817
Sep. 27, 2007 (US) PCT/US07/20818
Sep. 27, 2007 (US) PCT/US2007/020816

(51) Int. Cl. A61M 37/00

(52) U.S. Cl. 604/20

(57) ABSTRACT

A method for treatment of cancerous tissue in the upper respiratory system including the steps of: injecting HPPH in a physiologically compatible medium into a patient having the cancerous tissue at a level of 3 through 5 mg/m² of body surface area, waiting for a time period of 24 through 60 hours to permit preferential absorption of the HPPH into the cancerous tissue, and exposing the cancerous tissue to light at a wavelength of about 665±5 nm at an energy of about 75 to about 200 Joules/cm.

### CROSS REFERENCE TO RELATED PATENT APPLICATIONS

This application claims priority from PCT Application PCT/U.S.07/20818, filed Sep. 27, 2007 which in turn claims priority from U.S. Provisional Application No. 60,967,652, filed Sep. 6, 2007; U.S. Provisional Application No. 60/879,474, filed Jan. 9, 2007 and U.S. Provisional Application No. 60/879,435, filed Jan. 9, 2007; and PCT Application PCT/U.S.07/20817, filed Sep. 27, 2007 which in turn claims priority from U.S. Provisional Application 60/879,474, filed Jan. 9, 2007; and PCT Application PCT/US2007/020816, filed Sep. 27, 2007 which in turn claims priority from U.S. Provisional Application 60/879,435, filed Jan. 9, 2007.

### STATEMENT REGARDING FEDERALLY SPONSORED RESEARCH

This invention was made with funding from the National Institute of Health Grant Number NIH (1R21 CA109914-01, CA 55792, and CAPO155791). The United States Government may have certain rights in this invention.

### BACKGROUND OF THE INVENTION

The National Cancer Institute estimated that in 2007 there would be 11,300 new cases of laryngeal cancer and 3,660 deaths. In addition it is predicted that 34,360 new cases of oral and pharyngeal cancers will occur in 2007 (NCI Surveillance, Epidemiology and End Results Program, 2007). Pfister et al. have published the American Society of Clinical Oncology Clinical Practice Guidelines for preservation of the larynx in the treatment of laryngeal cancer. These were developed by an expert panel by review of literature available through 2005. Their recommendation for patients with T1 or T2 laryngeal cancer, with rare exceptions is that they should be treated with intent to preserve the larynx. The methods recommended include radiation or larynx preserving surgery depending on patient factors. They recommend that these treatments not be combined since single treatment is effective for limited stage, non-invasive cancer of the larynx and functional outcomes may be compromised with combined modality treatment.

Photodynamic therapy (PDT) with porfimer sodium has been approved by health agencies in Canada (bladder, esophageal cancer), Europe (esophageal, lung cancer) and in the United States (early and advanced cancers of the lung and advanced esophageal cancer, high grade dysplasia of the esophagus).

A review of published and non-published sources not necessarily prior art to the present invention, indicates that the use of porfimer sodium at its optimized dose level of 2 mg/kg and activation at 630±5 nm, and light 100 to 250 joules/cm resulted in destruction of high grade dysplasia and replacement of 75 to 80% of Barrett's mucosa with normal esophageal mucosain all patients treated (100 patients). Complete ablation of Barrett's mucosa was observed in 43% of patients. Of these, 8% achieved complete ablation of Barrett's mucosa with PDT treatment only, while 35% required thermal ablation to destroy small residual islands of abnormal mucosa Esophageal strictures occurred in 34% of all patients treated. The treatment also results in damage to surrounding normal tissue.

The use of HPPH for treatment of obstructive esophageal cancer has been described. (Optical Methods for Tumor Treatment and Detection: Mechanisms and Techniques in Photodynamic Therapy IX, Thomas Dougherty, Editor, Proceedings of SPIE Vol. 3909 (2000). This document does not describe effects on high grade dysplasia in Barrett's esophagus, Barrett's metaplasia itself or cancers in the head and neck.

In PDT, porfimer sodium is also used in numerous ‘off label’ indications including advanced, recurrent or refractory cancers of the head and neck, pleural cavity, brain, prostate, colon, skin and others. While often producing partial responses often resulted in eventual recurrence. Therefore, PDT was considered of limited benefit to patients with advanced disease. However, several small series of patients with superficial T1 tumors have been reported. Wenig et al. reported a 77% complete response in 26 patients with T1 recurrent lesions (9 oral cavity, 10 oropharynx, 2 nasopharynx, 2 neck and 1 each in the maxillary sinus, larynx and parotid gland) with follow up of 6 to 51 months. Biel reported treatment of 336 patients, with tumors at various locations. In that series 117 patients had squamous cell carcinoma of the larynx and were treated for cure. Three patients had recurrent CIS lesions, 92 had T1N0 carcinomas of the true vocal cord of which 25 were radiation failures and 15 patients had T2N0 lesions of the true vocal cord, of which 8 were radiation failures. All patients underwent one micro lens PDT treatment and T2 tumors also received implant PDT. With follow-up to 189 months (mean 84 months) there were 10 recurrences. All were salvaged with PDT, surgery or radiation. Adverse reactions were edema and erythema.

The natural history of this dysplasia is that over 30% of these patients would have developed invasive carcinoma, likewise the majority of patients with CIS would have developed an invasive cancer within a short period of time. Therefore with the eradication of dysplasia and CIS using PDT, initial studies indicate that development of invasive carcinoma can be prevented. In addition, other options such as surgery and radiotherapy which have permanent tissue effects and morbidity would not have to be utilized as first line therapy in these patients with minimal disease. Surgery and radiotherapy can be used in the future if PDT fails or if new cancers develop that are not amenable to PDT treatment (these patients experience a higher incidence of oral malignancy).

While PDT with the FDA-approved drug porfimer sodium is a highly effective treatment modality, the persistence of porfimer sodium in skin and associated photosensitization necessitates complete protection from sunlight and other sources of bright light for periods up to 90 days. Further, porfimer sodium is activated by light at 630 nm, which is suboptimal for tissue penetration. In addition, in tissues found in the upper respiratory system, side effects to normal tissue proximate to a tumor site has been greater than desirable, e.g. edema and normal tissue destruction, sometimes contributing to weeks for recovery and in some cases possibly causing permanent injury to normal proximate tissue. These drawbacks have led to a search for other photosensitizers without these limitations. This is especially important with respect to cancers that interfere with respiration, e.g. nose, mouth, pharynx, larynx and trachea. Temporary interference with digestive function can be tolerated since the body can cope without food input for a significant period of time and additionally, intravenous feeding can be readily done for temporary nutrition. The same case can of course not be made when respiration is involved since interference with respiration for even a short period is exceedingly distressful and in extreme cases can be fatal. In the case of cancers that involve the upper respiratory system it is therefore exceedingly important that damage to normal tissue be avoided.

‘upper respiratory system’ as used in the context of this invention means the nasal cavity, sinuses, oral cavity, pharynx, larynx, trachea, vocal cord and associated structures.

Serious cancers, especially in the upper respiratory system are usually squamous cell type carcinomas that are almost always fatal unless treated and even then treatment is not always effective and is often disfiguring.

For the last several years porphyrin-based compounds have been used for the treatment of cancer by photodynamic therapy (PDT). The concentration of certain porphyrins and related tetrapyrrolic systems is higher in malignant tumors than in most normal tissues and that has been one of the main reasons for using these molecules as photosensitizers. Some tetrapyrrole-based compounds have been effective in a wide variety of malignancies, including skin, lung, bladder, and esophagus. There have, however been associated problems with their use including skin phototoxicity, normal tissue damage due to the PDT treatment itself, and insufficient depth of penetration.

The precise mechanism(s) of PDT are unknown; however, in vivo animal data suggests that both direct cell killing and loss of tumor vascular function play a significant role.

A relatively new and well tested tetrapyrrolic compound is 2-(1-hexyloxyethyl)-2-devinyl pyropheophorbide-a (HPPH). HPPH, as used herein, means 2-(1-hexyloxyethyl)-2-devinyl pyropheophorbide-a as a free acid, the acid salt form as well as the various esters. This compound is tumor-avid and has undergone Phase I/II human clinical trials at the Roswell Park Cancer Institute in Buffalo, N.Y.

### BRIEF DESCRIPTION OF THE INVENTION

In accordance with the invention, we have surprisingly discovered that HPPH, like porfimer sodium also ablates cancers that involve epithelial type tissue in the upper respiratory system, when combined with exposure of such tissue to light at 665±5 nm. However, it has been surprisingly discovered that HPPH accomplishes the desired result at lower dosages and importantly with less damage to normal tissues than does porfimer sodium. HPPH is effective at doses of only 0.08 to 0.13 mg/kg of body weight (3.5 mg/m2 of body surface area) versus a minimum of 2 mg/kg of body weight for porfimer sodium. Further, it has been surprisingly discovered that HPPH concentrates in a much greater amount in tumors in epithelial type tissue than in normal tissue when compared with porfimer sodium thus leading to less normal tissue damage at effective treatment levels.

The invention is a method for treatment of cancerous tissue in the upper respiratory system including the steps of:

injecting HPPH in a physiologically compatible medium into a patient having the cancerous tissue to provide a dose level of 3 through 5 mg/m² of body surface area,

waiting for a time period of 24 through 60 hours to permit preferential absorption of the HPPH into the cancerous tissue, and

exposing the cancerous tissue to light at a wavelength of about 665±5 nm at an energy of about 75 to about 200 Joules/cm. The invention also includes HPPH for use in the above described method.

### DETAILED DESCRIPTION OF THE INVENTION

As previously discussed, it has now been surprisingly discovered that HPPH concentrates in normal epithelial tissue to a much less extent than the commonly used porfimer sodium and thus logically permits reduced tissue damage in normal epithelial tissues due to treatment exposure when compared with porfimer sodium.

PDT using a novel photosensitizer (HPPH), described in detail below, is being studied for treatment of high grade dysplasia (HGD) in Barrett's esophagus as described in PCT Patent Application PCT/U.S.07/20817, obstructive lung cancer and early stage lung cancer. The incentive for these studies has been the high efficacy comparable to porfimer sodium and lack of prolonged cutaneous photosensitivity (less than 1-2 weeks compared to 4-6 weeks for porfimer sodium) which has severely limited the use of porfimer sodium in Photodynamic Therapy.

HPPH, i.e. 2-(1-hexyloxyethyl)-2devinyl pyropheophorbide-ahas the following formula:

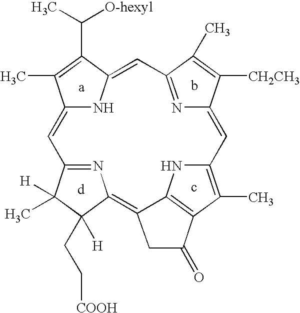

and includes the acid form, the various salts and alkyl esters thereof and may be prepared as set forth in U.S. Pat. Nos. 5,198,460 and 5,314,905 reissued as RE39094 and RE38994 respectively, all of which are incorporated herein by reference.

Tetrapyrollic photosensitizer compounds such as the photosensitizer porfimer sodium, sold under the trademark PHOTOFRIN™ and HPPH concentrate in most tumor tissue but it has now been unexpectedly discovered that the ratio of concentration in normal tissue to the concentration in abnormal tissue is markedly lower with HPPH than with porfimer sodium.

Responses of normal tissue and malignant tissue to PDT treatment are essentially the same under the same conditions, i.e. same light frequency, light dose, treatment time and same concentration of the same photodynamic compound in the cell. Light frequency is generally fixed by the absorption characteristics of the photodynamic compound used and treatment time is selected at the most optimum time for complete ablation of malignant tissue.

Damage to normal tissue up to now has been has been minimized by attempting to minimize the amount of normal tissue which can result in incomplete treatment as cancerous cells may extend beyond the treatment field. Since other photosensitizers are no more selective than is porfimer sodium for PDT treatment, there was no reason to select a PDT agent other than FDA approved porfimer sodium for purposes of obtaining less normal tissue damage as a result of treatment exposure.

Now, in accordance with the present invention, damage to normal tissue can be reduced by using a photodynamic compound that concentrates better in malignant tissue than normal tissue compared to porfimer sodium thus differentiating conditions between normal cells and malignant cells in the zone of treatment.

In accordance with the present invention, it has now been discovered that less HPPH concentrates in normal epithelial tissue, e.g. skin, than in malignant tissue by almost a factor of ten, i.e. amount in normal tissue over amount in malignant tissue found to be about 0.14. In such a case, at an optimal malignant cell concentration for ablation of malignant tissue of e.g. 4 mg/m² (about 0.1 mg/kg), the concentration in normal tissue would only be about 0.56 mg/m² (about 0.015 mg/kg), well below a concentration permitting serious effect upon normal tissue. By contrast, the commonly used porfimer sodium concentrates in normal epithelial tissue, e.g. skin at a rate almost equal to the amount in malignant tissue, i.e. amount in normal tissue over amount in malignant tissue found to be about 0.93. In such a case with porfimer sodium, at an optimal malignant cell concentration for use of porfimer sodium, e.g. 5 mg/kg, the concentration in normal tissue would be about 4.65 mg/kg, almost the same as in malignant tissue. It is thus logical that the use of porfimer sodium in PDT would cause normal tissue to be completely destroyed in the zone of treatment requiring healing of normal tissue within the treatment zone by regeneration of normal tissue from areas surrounding the treatment zone.

This is an important distinction between porfimer sodium and HPPH making HPPH uniquely qualified for treatment of carcinomas in epithelial type tissue in the upper respiratory area.

A table showing relative concentrations in normal tissue and cancer tissue for various tissue types is shown below. Ratios are shown in the form of concentration in normal tissue/concentration in tumor tissue. Lower numbers thus represent less PDT agent (HPPH or porfimer sodium) in normal tissue relative to concentration in tumor tissue. Lower concentrations in normal tissue would be expected to result in less tissue damage at tumor concentrations sufficient to destroy tumor tissue.

Ratio of Normal Tissue levels of two photo
sensitizers in Mice to Tumor Levels
Photofrin vs. HPPH

| | HPPH^{#} | PHOTOFRIN^{#} |
|---|---|---|
| Liver | 1.9 | 7 |
| Adrenals | 0.95 | 6.5^{###} |
| Spleen | 0.14 | 3.1^{###} |
| Kidney | 0.12 | 4 |
| Urinary Bladder | 0.11 | 5.9^{###} |
| Pancreas | 0.11 | 4.3^{###} |
| Muscle | 0.095 | 0.33 |
| Brain | 0.01 | 0.05^{###} |
| Skin | 0.14 | 0.93 |
| Lung | 2.7 | 1.7 |

^{#}Absolute Tumor Levels

| | |
|---|---|
| 3.0 mg/g | 1.05 mg/g |

(^{###}normalized to 5 mg/kg from data obtained at 27 mg/kg)

Ratios were obtained by dividing actual amounts of the photosensitizer in a given tissue by the amount in the tumor.

Data at 24 h post injection were used since this is the time the mice are treated by light, generally from a laser at non-thermal dose rates.

HHPH in patients is infused over one hour in a physiologically compatible medium

The concentration of HPPH in solution is preferably 0.8 through 1.5 mg/ml in medium and the medium is preferably 0.1% polysorbate 80, 2% ethyl alcohol and 5% glucose in normal saline.

Exposure is accomplished using a fiber optic carrying non-thermal laser light emitted by a laser. The laser may be any suitable laser emitting light at the wavelength and energy desired, e.g. a dye or diode. Exposure may be adjusted by length of time of exposure and/or adjustment of light intensity.

The following examples of preliminary results of treatment of cancers of the upper respiratory system by HPPH-PDT, illustrate the present invention.

### Example 1

A 65 year old male presented with a squamous cell carcinoma on his larynx. He refused surgery and radiation therapy and chose photodynamic therapy treatment using HPPH (2-[1-hexyloxyethyl]-2-devinyl pyropheophorbide a). He was infused over one hour with HPPH at 4 mg/m² (about 0.1 mg/kg). The following day he received non-thermal low power laser light treatment (150 mw/cm) at 665 nm in order to activate the photodynamic process. The light was delivered by a single quartz fiber treaded through a laryngoscope. He received 50 Joules/cm of the 665 nm light over 5.5 min. He developed transient edema and hoarseness of voice which was completely resolved in the first follow up examination 30 days after treatment. He experienced no skin photosensitivity. At the second follow up examination at 3 months he was found to have a complete response to treatment. No affect upon normal tissue was noted.

### Example 2

A 45 year old male presented with a squamous cell carcinoma of the floor of the mouth. He chose to have photodynamic therapy with HPPH (2-[1-hexyloxyethyl]-2-devinyl propheophorbide—a) rather than surgery or radiation therapy. He was infused with 4 mg/m² HPPH. The following day he received non-thermal low power laser light (150 mw/cm) at 665 nm in order to activate photodynamic process. The light was delivered by a single quartz fiber threaded through a laryngoscope. He received 50 Joules/cm of the 665 nm light over 5.5 min. He developed transient moderate pain at the treatment site controlled with analgesics. This was resolved when examined 30 days after treatment. He experienced no skin photosensitivity. At follow up at 6 months he was found to have a complete response to treatment confirmed by biopsy. No effect upon normal tissue was noted.

What is claimed is:

1. A method for treatment of cancerous tissue in the upper respiratory system comprising the steps of:
injecting HPPH in a physiologically compatible medium into a patient having the cancerous tissue at a level of 3 through 5 mg/m² of body surface area,
waiting for a time period of 24 through 60 hours to permit preferential absorption of the HPPH into the cancerous tissue, and
exposing the cancerous tissue to light at a wavelength of about 665±5 nm at an energy of about 75 to about 200 Joules/cm.

2. The method of claim 1 where the dose level of HPPH is 3.5 to 4.0 mg/m².

3. The method of claim 1 where the energy is from about 75 to about 150 Joules/cm.

4. The method of claim 1 where the cancerous tissue is cancerous tissue of epithelium.
